(19) United States
(12) Patent Application Publication
(10) Pub. No.: US 2002/0107444 A1
(43) Pub. Date: Aug. 8, 2002

(54) Image based size analysis

(76) Inventor: Doron Adler, Nesher (IL)

Correspondence Address: Eitan, Pearl, Latzer & Cohen-Zedek, Suite 210, One Crystal Park, 2011 Crystal Drive, Arlington, VA, 22202-3709, US

(21) Appl. No.: 09/739,379

(22) Filed: Dec. 19, 2000

(51) Int. Cl. A61B 5/00

(52) U.S. Cl. 600/424; 600/438; 600/109

(57) ABSTRACT

There is provided a method and system for calculating a size of an object in a gastrointestinal tract using images acquired by a moving imager. The method includes the steps of determining a distance traveled by the moving imager during capture of two of the images, calculating spatial coordinates of objects within the images using the distance, and calculating the size of an object from the spatial coordinates. The method and system may be used in the digestive tract with an endoscope or capsule.

### FIELD OF THE INVENTION

The present invention relates to a method and system for size analysis from two-dimensional images captured by a moving camera system.

### BACKGROUND OF THE INVENTION

One of the most important ways a physician has for analyzing a pathological condition is to examine the dimensions of the pathological entity. In the digestive tract, including the intestines, determination of size of an object within the tract can provide important information useful in diagnosing a condition and prescribing treatment. The use of size analysis and its importance in diagnosis can be seen from the numerous patents dealing with three-dimensional endoscopic imaging, such as U.S. Pat. Nos. 5,575,754, 4,651,201, 5,728,044, and 5,944,655. Many of the imaging systems discussed in the above patents use a plurality of imagers, imitating stereoscopic binocular vision in nature.

### SUMMARY OF THE INVENTION

There is provided, in accordance with one embodiment of the present invention, a method for calculating a size of an object in a gastrointestinal tract using images acquired by a moving imager. The method includes the steps of determining a distance traveled by the moving imager during capture of two of the images, calculating spatial coordinates of each of the pixels by using the distance, and calculating the size of the object from the spatial coordinates.

In one embodiment, the distance traveled by the imager is non-negligible as compared to the distance between the moving imager and the objects. In one embodiment, the moving imager includes a single camera. In one embodiment, the moving imager is an in vivo imager and is used in an endoscope.

There is provided, in accordance with another embodiment of the present invention, a system for calculation of object size by conversion of two-dimensional images, where the two-dimensional images are acquired by a moving imager. The system includes a distance-detecting unit for determining a distance traveled by the moving imager during the capture of two of the images, and at least one processor for generating spatial coordinates of objects within the images. The processor uses the distance obtained by the distance-detecting unit, and converts the spatial coordinates into a size calculation of the object.

In one embodiment, the imager is an in vivo imager, and has a single camera. In one embodiment, the distance-detecting unit is a sensor. In one embodiment, the sensor is a position sensor which has three receivers which receive signals from a transmitter in communication with the camera system, the receiver in communication with a unit for determining the position of the camera system. The position sensor may be an induction coil. In another embodiment, the sensor is an image analyzer which can analyze the optical flow of an image. In another embodiment, the sensor is a velocity sensor, which may be an accelerometer or an ultrasound transducer. In one embodiment, the system may be used in an endoscope.

There is provided, in accordance with another embodiment of the present invention, a swallowable capsule for calculating a size of an object in a gastrointestinal tract. The capsule includes an image receiver for receiving images within the gastrointestinal tract, a distance detecting unit for determining a distance traveled by the capsule during reception of two images, and a processor for generating spatial coordinates of an object found within the images and converting the spatial coordinates into a size calculation of the object.

### BRIEF DESCRIPTION OF THE DRAWINGS

The present invention will be understood and appreciated more fully from the following detailed description taken in conjunction with the drawings in which:

FIG. 1 is a schematic illustration of a prior art in vivo camera system;

FIG. 2 is a schematic illustration of the in vivo capsule of FIG. 1 transiting part of the gastro-intestinal lumen;

FIG. 3 is a block diagram illustration of a system according to one embodiment of the present invention;

FIG. 4 is a flow chart illustration of the method used by the system shown in FIG. 3; and

FIG. 5 is a schematic illustration showing how spatial coordinates are determined according to the present invention.

Similar elements in the Figures are numbered the same throughout.

### DETAILED DESCRIPTION OF THE INVENTION

Various in vivo measurement systems are known in the art. They typically include swallowable electronic capsules which collect data and which transmit the data to a receiver system. These intestinal capsules, which are moved through the digestive system through the action of peristalsis, are often called “Heidelberg” capsules and are utilized to measure pH, temperature (“Coretemp”) or pressure throughout the intestines. They have also been used to measure gastric residence time, which is the time it takes for food to pass through the stomach and intestines.

These intestinal capsules typically include a measuring system and a transmission system, where the transmission system transmits the measured data at radio frequencies to a receiver system, The receiver system is usually located outside the body. Other systems can store all the data within a storage device in the capsule. The data can then be read after the capsule exits the gastro-intestinal (GI) tract.

U.S. Pat. No. 5,604,531, assigned to the common assignee of the present application and incorporated herein by reference, teaches an in vivo camera system, which is carried by a swallowable capsule. The in vivo video camera system captures and transmits images of the GI tract while the capsule passes through the GI lumen. In addition to the camera system, the capsule contains an optical system for imaging an area of interest onto the camera system and a transmitter for transmitting the video output of the camera. The capsule can pass through the entire digestive tract and operate as an autonomous video endoscope. It images even the difficult to reach areas of the small intestine.

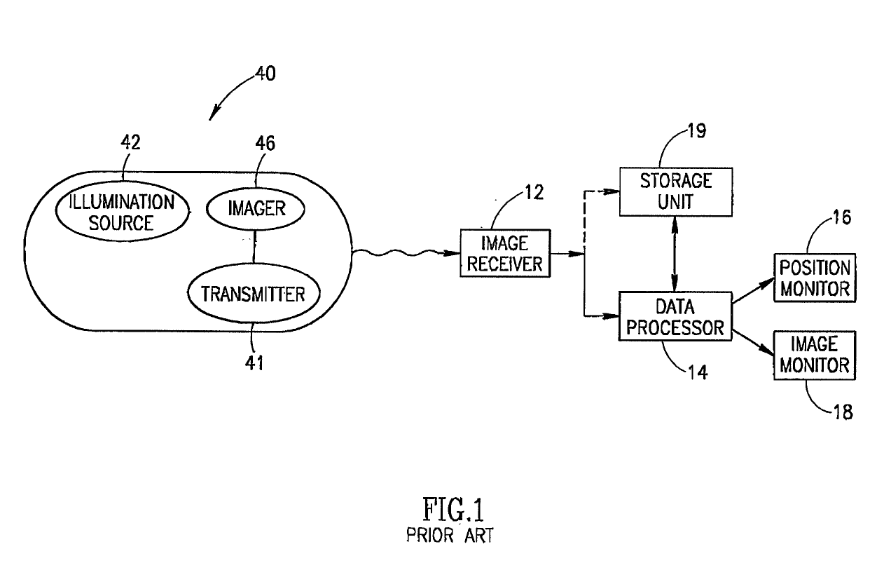

Reference is made to FIG. 1, which shows a schematic diagram of the system, described in U.S. Pat. No. 5,604,531. The system comprises a capsule 40 having an imager 46, an illumination source 42, and a transmitter 41. Outside the patients body are an image receiver 12 (usually an antenna array), a storage unit 19, a data processor 14, an image monitor 18, and a position monitor 16. While FIG. 1 shows separate monitors, both an image and its position can be presented on a single monitor.

Imager 46 in capsule 40 is connected to transmitter 41 also located in capsule 40. Transmitter 41 transmits images to image receiver 12, which sends the data to data processor 14 and to storage unit 19. Data processor 14 analyzes the data and is in communication with storage unit 19, transferring frame data to and from storage unit 19. Data processor 14 also provides the analyzed data to image monitor 18 and position monitor 16 where the physician views the data. The image monitor presents an image of the GI lumen and the position monitor presents the position in the GI tract at which the image was taken. The data can be viewed in real time or at some later date. In addition to revealing pathological conditions of the GI tract, the system can provide information about the location of these pathologies.

The present invention relates to a method and system of size analysis by converting two-dimensional images, captured by a moving in-vivo video camera system, such as that of FIG. 1, into three-dimensional representations. This conversion is done by only one camera or imager, and is based on knowing the velocity of the camera system when it captures the frames being converted.

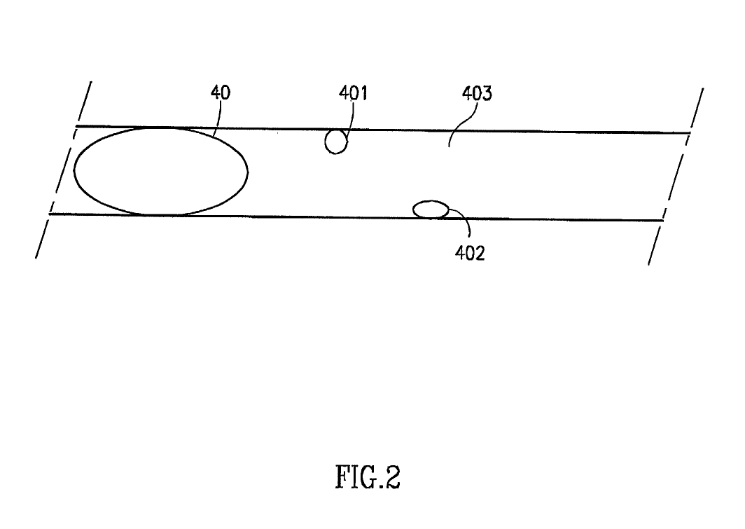

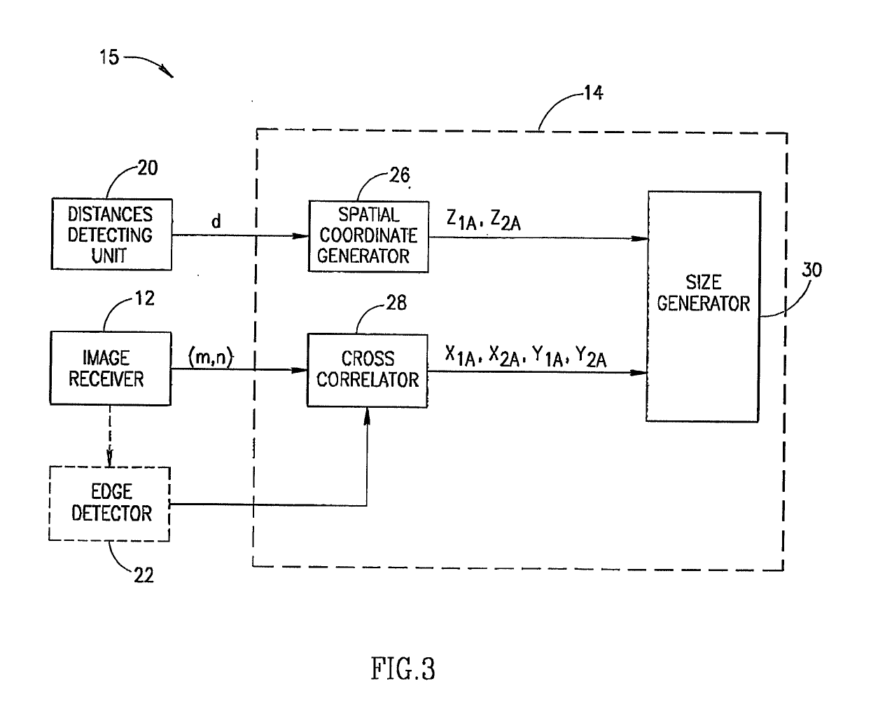

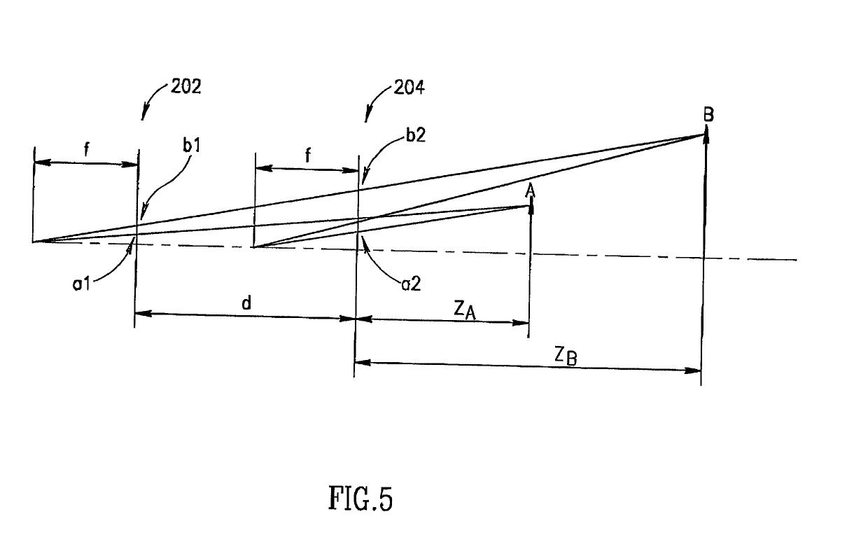

Reference is now made to FIGS. 2 and 3, which illustrate a video capsule 40 inside the gut approaching two objects, and a system 15 for determining the size of one of the objects, according to one embodiment of the present invention. In FIG. 2, video capsule 40 is shown approaching a first object 401 and a second object 402 in GI lumen 403. Using two, usually, but not necessarily, consecutive images captured by capsule 40 and the known speed of capsule 40, size analysis based on three dimensional representations of objects 401 and 402 can be done, as will be discussed with regard to FIG. 5 below.

In FIG. 3, system 15 comprises a distance-detecting unit 20, an image receiver 12 and a processor 14. Processor 14 comprises a spatial coordinate generator 26, a cross correlator 28 and a size generator 30. In one embodiment, distance-detecting unit 20 is a position detector. In one embodiment, distance-detecting unit 20 obtains a distance measurement d by measuring and integrating a velocity, as will be described hereinbelow. Processor 14 is a standard PC accelerator board, high performance PC, multiprocessor PC or any other serial or parallel high performance processing machine. Optionally, system 15 may comprise an edge detector 22. Any edge detector used in conventional image analysis can be used, such as the following sliding window filter:

$$[ \begin{matrix}- 1 & 0 & - 1 \\ 0 & 4 & 0 \\ - 1 & 0 & - 1\end{matrix} ]$$

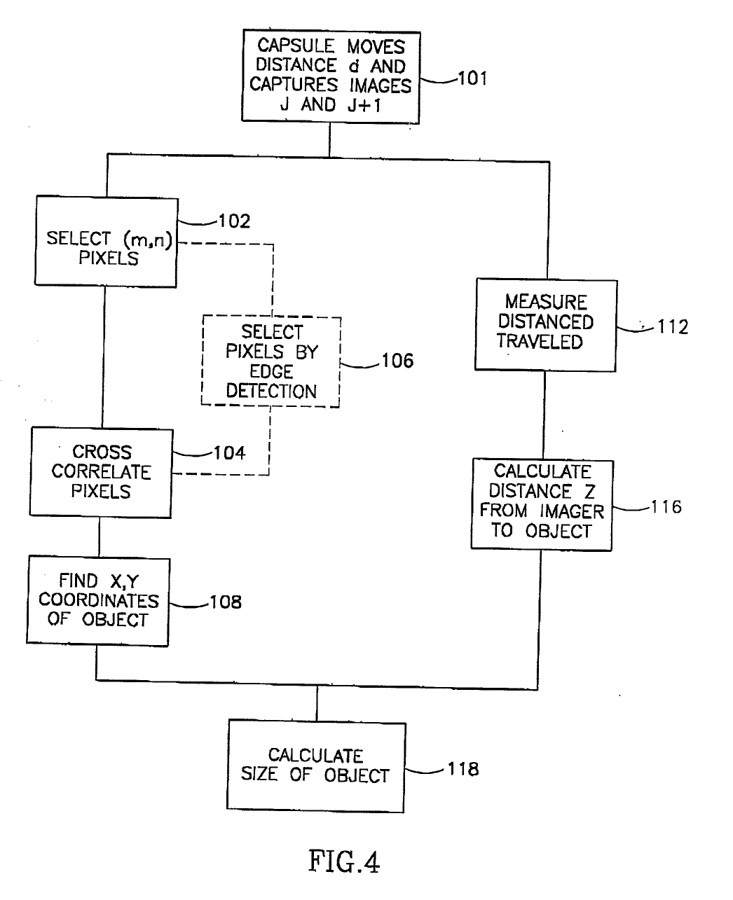

Reference is now made to FIG. 4 which is a flow chart diagram illustrating a general method for generating size measurements from two-dimensional images according to one embodiment of the present invention. Steps of FIG. 4 may be accomplished using system 15 of FIG. 3. First, image receiver 12 (within a moving in vivo video camera system such as the one described in FIG. 1) captures (step 101) images periodically, such as every 100-1000 ms. In one embodiment, the images are captured every 500 ms. Data processor 14 divides received images into a grid of pixels, and selects (step 102) pixels for analysis. As in other imaging applications, the number of pixels determines the resolution of the image For purposes of this discussion, the images are divided into m×n pixels.

Next, cross correlator 28 calculates (step 104) an xy cross correlation function between the intensities Iⱼ and Iⱼ₊ₙ of image j and image j+n, thereby identifying corresponding pixels in images j and j+1. The value n is usually, but not necessarily, 1. Henceforth, the second frame will be designated as j+1, with the understanding that n can also be greater than 1.

The correlation can be done for each of the m×n pixels created in images j, and j+1. However, in another embodiment, edge detector 22 selects (step 106) pixels for cross correlation, thereby selecting an object. In one embodiment, only pixels whose edges exceed a certain predetermined threshold value are selected for correlation.

While the cross correlation can be done on a pixel by pixel basis, more often, it is performed on parts of the image, such as sets of 8×8 pixels. The latter approach can be used to minimize computation time.

In one typical cross correlation function, the cross correlation coefficient C_{xy}is given by:

$${C}_{xy} = {\sum }_{m} {\sum }_{n} {I}_{j} ( m , n ) {I}_{j + 1} ( m + x , n + y )$$

where Iⱼ(m, n) and Iⱼ₊₁(m, n) are the intensity values of pixel (m, n) in images j and j+1 respectively. The vector (x, y) can be considered the displacement vector from pixel (m, n) in going from pixel (m, n) to pixel (m+x, n+y). The maximum of the cross correlation function indicates the most probable location of correspondence between the pixels of images j and j+1. A suitable cross correlation function is included in Matlab, a standard mathematics package for computers.

The results of the cross correlation provide x and y coordinates for a specific point. If the cross correlation is performed for four edges of an object on images j and j+1, an entire two-dimensional set of spatial coordinates is obtained (step 108). Thus, for object A, x_{1A}, x_{2A}, y_{1A} and y_{2A} are known.

The determination of the z coordinates for object A is based on knowing the distance traversed by imager 46 while it moves through the GI tract capturing images j and j+1. In one embodiment, distance-measuring unit 20 measures the velocity of imager 46 using an accelerometer and an integrator. The accelerometer may be, for example, the ADXL50 model from Analog Devices. It is readily evident that, in addition to an accelerometer, any sensor that can determine the velocity of the capsule could also be used. Such sensors include, but are not limited to, induction coils (as described in U.S. Pat. No. 4,431,005, incorporated herein by reference) and ultrasound transducers. For example, if an induction coil is located in the capsule and the patient is placed in a magnetic field, a current would be produced by the coil with a magnitude proportional to the velocity of the capsule. Similarly, ultrasound transducers, such as those used in conventional medical ultrasound devices, can be used as an external sensor to track the movement of the capsule and standard electronics could be used to convert the data to velocities.

In another embodiment, the change of position of the capsule while capturing two images can be used to determine the distance traveled by the capsule during the time interval between the images. Signals sent by a transmitter within the capsule and received by receivers outside the body can be used to locate the position of the capsule. A suitable system for determining capsule location is one described in U.S. Provisional Application Serial Number 60/187,885 assigned to the common assignee af the present application and incorporated herein by reference.

In yet another embodiment, conventional image analysis techniques can be used to analyze the optical flow of the images. On the basis of the smear pattern of the images, velocity or distance can be determined. Once the velocity is known, an integrator calculates (step 112) the distance traveled by imager 46 from the time of capture of image j to the time of capture of image j+1. This distance value is used in determining (step 116) the z coordinate of object A, as described in two possible methods hereinbelow.

The first method described hereinbelow is adapted from a method discussed in Machine Vision: Theory, Algorithms, Practicalities, E. R. Davies, Academic Press 1996, pp. 441-444, incorporated herein by reference. Davies describes how a camera, when moving along a baseline, sees a succession of images. Depth information can be obtained by analyzing the object features of two images.

In general, the discussion by Davies uses far-field approximations; he discusses systems where the distance traveled by a camera between images is far smaller than the distance to the object. That is a condition that does not apply to in viva video camera systems imaging the GI tract. In vivo video camera systems usually move distances that are non-negligible in size when compared to the distances between the camera and objects being imaged. Because far field approximations are not valid for in vivo video camera systems, images of two objects are required, where one object serves as a reference object.

Reference is now made to FIG. 5, which shows a geometric illustration of the basis for calculating the z coordinate of two objects A and B. It should be noted that the z coordinate represents the distance from imager 46 to each of the objects, denoted z_{A} and z_{B} respectively.

As mentioned above, imager 46 moves a certain distance d from the capture of the first image 202 to the capture of the second image 204. Thus, the distance between images 202 and 204 is distance d. In addition, there is a certain focal length f, which is the lens focal length. While focal length f is used in the derivation of the following equations, it is eventually eliminated and its value does not need to be known explicitly.

The projections of objects A and B on each of the images 202 and 204 in the y direction are shown in FIG. 5 and are denoted a1, b1, a2 and b2, respectively. These values are obtained from the pixel information stored in storage unit 19, and correspond to the n value of each m×n pixel. Thus, for example, a1 represents the X value of object A as it was acquired in time t1 (X_{1A}) and a2 represents the X value of object A as it was acquired in time t2 (X_{2A}). Accordingly, b1 represents the X value of object B as it was acquired in time t1 (X_{1B}) and b2 represents the X value of object B as it was acquired in time t2 (X_{2B}).

The actual values for a1, a2, b1, and b2 are calculated by image processor 14 (step 108 of FIG. 4) from the size of the sensor and image pixel data stored in storage unit 18. Thus, if the sensor has a length L, and there are m pixels along the X axis, then an object whose length is p pixels will have an actual size of: L * P/m.

Using similar triangles, it can be shown that the following relationship exists;

Z_{b}(1−T_{b})=Zₐ(1−Tₐ)+d(T_{b}−Tₐ)

where Tₐ and T_{b} are defined as:

Tₐ=a₁/a₂

T_{b}=b₁/b₂

Thus, the z coordinate for object A as a function of the z coordinate for object B can be obtained. Spatial coordinate processor 26 calculates (step 116) the z values for two points on object A (z_{1A} and z_{2A}) corresponding to the two edges of object A Accordingly, xyz spatial coordinates are known for object A. Size analyzer 30 then calculates (step 118) the size of object A by subtracting each of the axis coordinates from each other. Thus, x_{A}=x_{2A}−x_{1A}; y_{A}=y_{2A}−y_{1A}; and z_{A}=z_{2A}−z_{1A}, resulting in values for length, width and height, respectively, of object A.

Alternatively, another method can be used to calculate z_{A}, which is based on the following relationships:

A/a₁=(Z_{A}+d+f)/f

A/a₂=(Z_{A}+f)/f

From those two equations the following can be calculated:

A*f=(Z_{A}+d+f)/a₁=(Z_{A}+f)*a₂

Leading to

Z_{A}*a₁−Z_{A}*a₂=f*a₂−(d+f)*a₁=f*(a₂−a₁)−d*a₁

Finally,

Z_{A}=d*a₁/(a₂−a₁)−f

Thus, if the focal length of the camera is known, only one object is needed for calculation. Size of the object is calculated as above. Image processor 14 sends any selected size-data to image monitor 18 for display.

The procedure described hereinabove can be performed as a post-processing step, or, with adequate computational capability, it can be done in real time, allowing the user to choose specific images for processing.

It should be evident that while FIG. 5 shows a one-dimensional object, (e.g. a line), here positioned along the X-axis, symmetry considerations can be used in an analogous manner to obtain the Y coordinate, where the Y-axis is perpendicular to the plane of the paper.

It will be appreciated by persons skilled in the art that the present invention is not limited to what has been particularly shown and described hereinabove. Rather the scope of the present invention is defined only by the claims that follow:

What is claimed is:

1. A method for calculating a size of an object in a gastrointestinal tract using images acquired by a moving imager, wherein said method comprises the following steps:
determining a distance traveled by said moving imager during capture of two of said images;
calculating relative spatial coordinates of objects within said images using said distance; and
calculating the size of one of said objects from said spatial coordinates.

2. A method according to claim 1 wherein said distance is non-negligible as compared to a distance between said moving imager and said objects.

3. A method according to claim 1 wherein said moving imager comprises a single camera.

4. A method according to claim 1 wherein said moving imager is an in vivo imager.

5. A method according to claim 3 for use in an endoscope.

6. A system for calculation of object size by conversion of two-dimensional images, said two-dimensional images acquired by a moving imager, said system comprising:
a distance-detecting unit for determining a distance traveled by said moving imager during the capture of two of said images; and
at least one processor for generating spatial coordinates of objects within said images, said processor using said distance obtained by said distance-detecting unit, whereby said at least one processor converts said spatial coordinates into a size calculation of said object.

7. A system according to claim 6 wherein said imager is an in vivo imager.

8. A system according to claim 7 wherein said in vivo imager comprises a single camera.

9. A system according to claim 6 wherein said distance-detecting unit is a sensor.

10. A system according to claim 9 where said sensor is a position sensor.

11. A system according to claim 10 wherein said position sensor comprises at least one receiver which receives signals from a transmitter in communication with said camera system, said receiver in communication with a means for determining the position of said camera system.

12. A system according to claim 10 wherein said at least one receiver includes three receivers.

13. A system according to claim 10 where said position sensor is an induction coil.

14. A system according to claim 9 where said sensor is an image analyzer which can analyze the optical flow of an image.

15. A system according to claim 9 wherein said sensor is a velocity sensor.

16. A system according to claim 15 wherein said velocity sensor is an accelerometer, data from said accelerometer being integrated to determine velocity.

17. A system according to claim 15 where said velocity sensor is an ultrasound transducer.

18. A system according to claim 7 for use in an endoscope.

19. A swallowable capsule for calculating a size of an object in a gastrointestinal tract, the capsule comprising:
an image-receiver for receiving images within the gastrointestinal tract;
a distance-detecting unit for determining a distance traveled by said capsule during reception of two of said images; and
a processor for generating spatial coordinates of at least one object found within said two images and for converting said spatial coordinates into a size calculation of said at least one object.

20. A capsule as in claim 19 wherein said distance-detecting unit is a sensor.

21. A capsule as in claim 20 wherein said sensor is a position sensor.

22. A capsule as in claim 21 wherein said position sensor comprises at least one receiver which receives signals from a transmitter in communication with said capsule, said receiver in communication with a means for determining the position of said capsule.

23. A capsule as in claim 22 wherein said at least one receiver includes three receivers.

24. A capsule according to claim 21 where said position sensor is an induction coil.

25. A capsule according to claim 20 wherein said sensor is an image analyzer which can analyze the optical flow of an image.

26. A capsule according to claim 20 wherein said sensor is a velocity sensor.

27. A capsule according to claim 26 wherein said velocity sensor is an accelerometer, data from said accelerometer being integrated to determine velocity.

28. A capsule according to claim 26 where said velocity sensor is an ultrasound transducer.
